(19) United States
(12) Patent Application Publication
(10) Pub. No.: US 2023/0024942 A1
(43) Pub. Date: Jan. 26, 2023

(54) COMPUTER ASSISTED SURGERY SYSTEM, SURGICAL CONTROL APPARATUS AND SURGICAL CONTROL METHOD

(71) Applicant: Sony Group Corporation, Tokyo (JP)

(72) Inventors: Christopher WRIGHT, London (GB); Bernadette ELLIOTT-BOWMAN, London (GB); Naoyuki HIROTA, Tokyo (JP)

(73) Assignee: Sony Group Corporation, Tokyo (JP)

(21) Appl. No.: 17/785,910

(22) PCT Filed: Nov. 5, 2020
(86) PCT No.: PCT/JP2020/041391

§ 371 Date: Jun. 16, 2022

(30) Foreign Application Priority Data
Dec. 23, 2019 (EP) 19219496.7

(51) Int. Cl. A61B 34/00; A61B 90/00; A61B 1/045; A61B 34/37; G06N 3/02

(52) U.S. Cl. CPC A61B 34/25; A61B 90/361; A61B 1/045; A61B 34/37; G06N 3/02; A61B 2034/305

(57) ABSTRACT

A computer assisted surgery system comprising an image capture apparatus, a display, a user interface and circuitry, wherein the circuitry is configured to: receive information indicating a surgical scenario and a surgical process associated with the surgical scenario; obtain an artificial image of the surgical scenario; output the artificial image for display on the display; receive permission information via the user interface indicating if there is permission for the surgical process to be performed if the surgical scenario is determined to occur.

### FIELD

The present disclosure relates to a computer assisted surgery system, surgical control apparatus and surgical control method.

### BACKGROUND

The “background” description provided herein is for the purpose of generally presenting the context of the disclosure. Work of the presently named inventors, to the extent it is described in the background section, as well as aspects of the description which may not otherwise qualify as prior art at the time of filing, are neither expressly or impliedly admitted as prior art against the present disclosure.

Some computer assisted surgery systems allow a computerised surgical apparatus (e.g. surgical robot) to automatically make a decision based on an image captured during surgery. The decision results in a predetermined process being performed, such as the computerised surgical system taking steps to clamp or cauterise a blood vessel if it determines there is a bleed or to move a surgical camera or medical scope used by a human during the surgery if it determines there is an obstruction in the image. Computer assisted surgery systems include, for example, computer-assisted medical scope systems (where a computerised surgical apparatus holds and positions a medical scope (also known as a medical vision scope) such as a medical endoscope, surgical microscope or surgical exoscope while a human surgeon conducts surgery using the medical scope images), master-slave systems (comprising a master apparatus used by the surgeon to control a robotic slave apparatus) and open surgery systems in which both a surgeon and a computerised surgical apparatus autonomously perform tasks during the surgery.

A problem with such computer assisted surgery systems is it is sometimes difficult to know what the computerised surgical apparatus is looking for when it makes a decision. This is particularly the case where decisions are made by classifying an image captured during the surgery using an artificial neural network. Although the neural network can be trained with a large number of training images in order to increase the likelihood of new images (i.e. those captured during a real surgical procedure) being classified correctly, it is not possible to guarantee that every new image will be classified correctly. It is therefore not possible to guarantee that every automatic decision made by the computerised surgical apparatus will be the correct one.

Because of this, decisions made by a computerised surgical apparatus usually need to be granted permission by a human user before that decision is finalised and the predetermined process associated with that decision is carried out. This is inconvenient and time consuming during the surgery for both the human surgeon and the computerised surgical apparatus. It is particularly undesirable in time critical scenarios (e.g. if a large bleed occurs, time which could be spent by the computerised surgical apparatus clamping or cauterising a blood vessel to stop the bleeding is wasted during the time in which permission is sought from the human surgeon).

However, it is also undesirable for the computerised surgical apparatus to be able to make automatic decisions without permission from the human surgeon in case the classification of a captured image is not appropriate and therefore the automatic decision is the wrong one. There is therefore a need for a solution to this problems.

### SUMMARY

According to the present disclosure, a computer assisted surgery system is provided that includes an image capture apparatus, a display, a user interface and circuitry, wherein the circuitry is configured to: receive information indicating a surgical scenario and a surgical process associated with the surgical scenario; obtain an artificial image of the surgical scenario; output the artificial image for display on the display; receive permission information via the user interface indicating if there is permission for the surgical process to be performed if the surgical scenario is determined to occur.

### BRIEF DESCRIPTION OF DRAWINGS

Non-limiting embodiments and advantages of the present disclosure will be best understood by reference to the following detailed description taken in conjunction with the accompanying drawings.

FIG. 1 schematically shows a computer assisted surgery system.

FIG. 2 schematically shows a surgical control apparatus.

FIG. 3A schematically shows the generation of artificial images of a predetermined surgical scenario for display to a human.

FIG. 3B schematically shows the generation of artificial images of a predetermined surgical scenario for display to a human.

FIG. 3C schematically shows the generation of artificial images of a predetermined surgical scenario for display to a human.

FIG. 4A schematically shows a proposal to adjust a field of view of an image capture apparatus for display to a human.

FIG. 4B schematically shows a proposal to adjust a field of view of an image capture apparatus for display to a human.

FIG. 5 shows a lookup table storing permissions associated with respective predetermined surgical scenarios.

FIG. 6 shows a surgical control method.

FIG. 7 schematically shows a first example of a computer assisted surgery system to which the present technique is applicable.

FIG. 8 schematically shows a second example of a computer assisted surgery system to which the present technique is applicable.

FIG. 9 schematically shows a third example of a computer assisted surgery system to which the present technique is applicable.

FIG. 10 schematically shows a fourth example of a computer assisted surgery system to which the present technique is applicable.

FIG. 11 schematically shows an example of an arm unit.

FIG. 12 schematically shows an example of a master console.

Like reference numerals designate identical or corresponding parts throughout the drawings.

### DESCRIPTION OF EMBODIMENTS

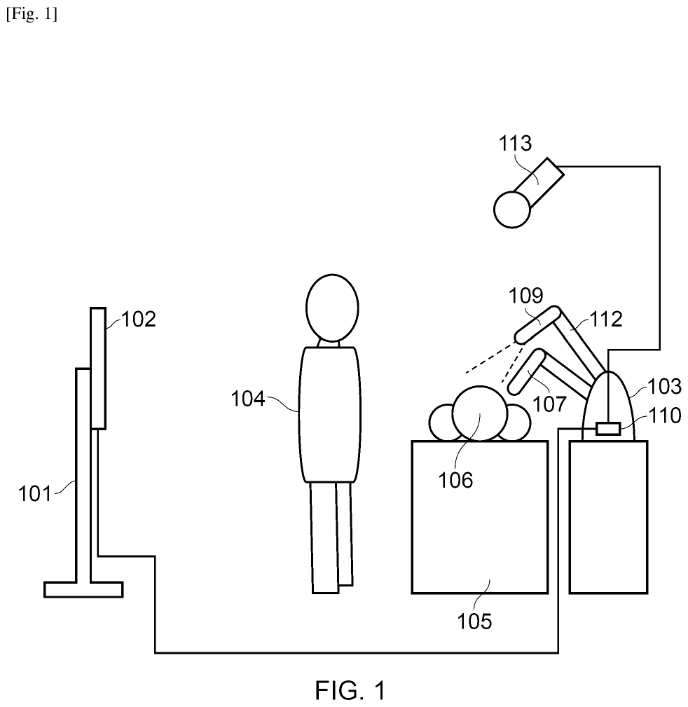

FIG. 1 shows surgery on a patient 106 using an open surgery system. The patient 106 lies on an operating table 105 and a human surgeon 104 and a computerised surgical apparatus 103 perform the surgery together.

Each of the human surgeon and computerised surgical apparatus monitor one or more parameters of the surgery, for example, patient data collected from one or more patient data collection apparatuses (e.g. electrocardiogram (ECG) data from an ECG monitor, blood pressure data from a blood pressure monitor, etc.—patient data collection apparatuses are known in the art and not shown or discussed in detail) and one or more parameters determined by analysing images of the surgery (captured by the surgeon's eyes or a camera 109 of the computerised surgical apparatus) or sounds of the surgery (captured by the surgeon's ears or a microphone 113 of the computerised surgical apparatus). Each of the human surgeon and computerised surgical apparatus carry out respective tasks during the surgery (e.g. some tasks are carried out exclusively by the surgeon, some tasks are carried out exclusively by the computerised surgical apparatus and some tasks are carried out by both the surgeon and computerised surgical apparatus) and make decisions about how to carry out those tasks using the monitored one or more surgical parameters.

It can sometimes be difficult to know why the computerised surgical apparatus has made a particular decision. For example, based on image analysis using an artificial neural network, the computerised surgical apparatus may decide an unexpected bleed has occurred in the patient and that action should be taken to stop the bleed. However, there is no guarantee that the image classification and resulting decision to stop the bleed is correct. The surgeon must therefore be presented with and confirm the decision before action to stop the bleed is carried out by the computerised surgical apparatus. This is time consuming and inconvenient for the surgeon and computerises surgical apparatus. However, if this isn't done and the image classification and resulting decision made by the computerised surgical apparatus is wrong, the computerised surgical apparatus will take action to stop a bleed which isn't there, thereby unnecessarily delaying the surgery or risking harm to the patient.

The present technique helps fulfil this need using the ability of artificial neural networks to generate artificial images based on the image classifications they are configured to output. Neural networks (implemented as software on a computer, for example) are made up of many individual neurons each of which activate under a set of conditions when the neutron recognises the inputs it is looking for. If enough of these neurons activate (e.g. neurons looking for different features of a cat such as whiskers, fur texture, etc.), then an object which is associated with those neurons (e.g. a cat) is identified by the system.

Early examples of these recognition systems suffer from a lack of interpretability, where an output (which attaches one of a plurality of predetermined classifications to an input image, e.g. object classification, recognition event or other) is difficult to trace back to the inputs which caused it. This problem has begun to be addressed recently in the field of AI interpretability, where different techniques may be used to follow the neural network's decision pathways from input to output.

One such known technique is feature visualization which is able to artificially generate the visual (or other data type, if another type of data is input to a suitable trained neural network for classification) features which are most able to cause activation of a particular output. This can demonstrate to a human what stimuli certain parts of the network are looking for.

In general, a trade off exists in feature visualization, where a generated feature which a neuron is looking for may be:
Optimized, where the generated output of the feature visualization process is an image which maximises the activation confidence of the selected neural network layers/neurons.
Diversified, where the range of features which activate the selected neural network layers/neurons can be exemplified by generated images.

These approaches have different advantages and disadvantages, but a combination will let an inspector of a neural network check what input features will cause neuron activation and therefore a particular classification output.

Feature visualization is used with the present technique to allow a human surgeon (or other human involved in the surgery) to view artificial images representing what the neural network of the computerised surgical apparatus is looking for when it makes certain decisions. Looking at the artificial images, the human can determine how successfully they represent a real image of the scene relating to the decision. If the artificial image appears sufficiently real in the context of the decision to be made (e.g. if the decision is to automatically clamp or cauterise a blood vessel to stop a bleed and the artificial image looks sufficiently like a blood vessel bleed which should be clamped or cauterised), the human gives permission for the decision to be made in the case that the computerised surgical apparatus makes that decision based on real images captured during the surgery. During the surgery, the decision will thus be carried out automatically without further input from the human, thereby preventing unnecessarily disturbing the human and delaying the surgery. On the other hand, if the image does not appear sufficiently real (e.g. if the artificial image contains unnatural artefacts or the like which reduce the human's confidence in the neural network to determine correctly whether a blood vessel bleed has occurred), the human does not give such permission. During the surgery, the decision will thus not be carried out automatically. Instead, the human will be presented with the decision during the surgery if and when it is made and will be required to give permission at this point. Decisions with a higher chance of being incorrect (due to a reduced ability of the neural network to correctly classify images resulting in the decision) are therefore not given permission in advance, thereby preventing problems with the surgery resulting from the wrong decision being made. The present technique therefore provides more automated decision making during surgery (thereby reducing how often a human surgeon is unnecessarily disturbed and reducing any delay of the surgery) whilst keeping the surgery safe for the patient.

Although FIG. 1 shows an open surgery system, the present technique is also applicable to other computer assisted surgery systems where the computerised surgical apparatus (e.g. which holds the medical scope in a computer-assisted medical scope system or which is the slave apparatus in a master-slave system) is able to make decisions. The computerised surgical apparatus is therefore a surgical apparatus comprising a computer which is able to make a decision about the surgery using captured images of the surgery. As a non-limiting example, the computerised surgical apparatus 103 of FIG. 1 is a surgical robot capable of making decisions and undertaking autonomous actions based on images captured by the camera 109.

The robot 103 comprises a controller 110 (surgical control apparatus) and one or more surgical tools 107 (e.g. movable scalpel, clamp or robotic hand). The controller 110 is connected to the camera 109 for capturing images of the surgery, to a microphone 113 for capturing an audio feed of the surgery, to a movable camera arm 112 for holding and adjusting the position of the camera 109 (the movable camera arm comprising a suitable mechanism comprising one or more electric motors (not shown) controllable by the controller to move the movable camera arm and therefore the camera 109) and to an electronic display 102 (e.g. liquid crystal display) held on a stand 101 so the electronic display 102 is viewable by the surgeon 104 during the surgery.

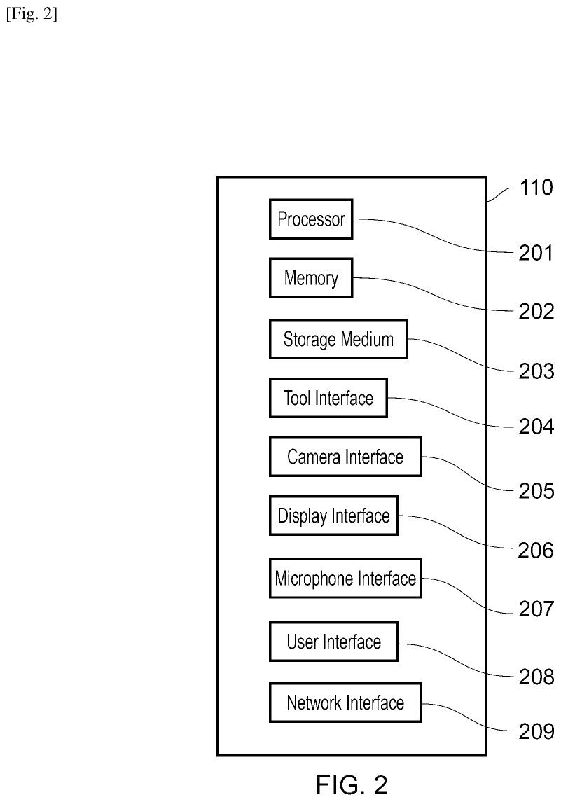

FIG. 2 shows some components of the controller 110.

The control apparatus 110 comprises a processor 201 for processing electronic instructions, a memory 202 for storing the electronic instructions to be processed and input and output data associated with the electronic instructions, a storage medium 203 (e.g. a hard disk drive, solid state drive or the like) for long term storage of electronic information, a tool interface 204 for sending electronic information to and/or receiving electronic information from the one or more surgical tools 107 of the robot 103 to control the one or more surgical tools, a camera interface 205 for receiving electronic information representing images of the surgical scene captured by the camera 109 and to send electronic information to and/or receive electronic information from the camera 109 and movable camera arm 112 to control operation of the camera 109 and movement of the movable camera arm 112, a display interface 202 for sending electronic information representing information to be displayed to the electronic display 102, a microphone interface 207 for receiving an electrical signal representing an audio feed of the surgical scene captured by the microphone 113, a user interface 208 (e.g. comprising a touch screen, physical buttons, a voice control system or the like) and a network interface 209 for sending electronic information to and/or receiving electronic information from one or more other devices over a network (e.g. the internet). Each of the processor 201, memory 202, storage medium 203, tool interface 204, camera interface 205, display interface 206, microphone interface 207, user interface 208 and network interface 209 are implemented using appropriate circuitry, for example. The processor 201 controls the operation of each of the memory 202, storage medium 203, tool interface 204, camera interface 205, display interface 206, microphone interface 207, user interface 208 and network interface 209.

In embodiments, the artificial neural network used for feature visualization and classification of images according to the present technique is hosted on the controller 110 itself (i.e. as computer code stored in the memory 202 and/or storage medium 203 for execution by the processor 201). Alternatively, the artificial neural network is hosted on an external server (not shown). Information to be input to the neural network is transmitted to the external server and information output from the neural network is received from the external server via the network interface 209.

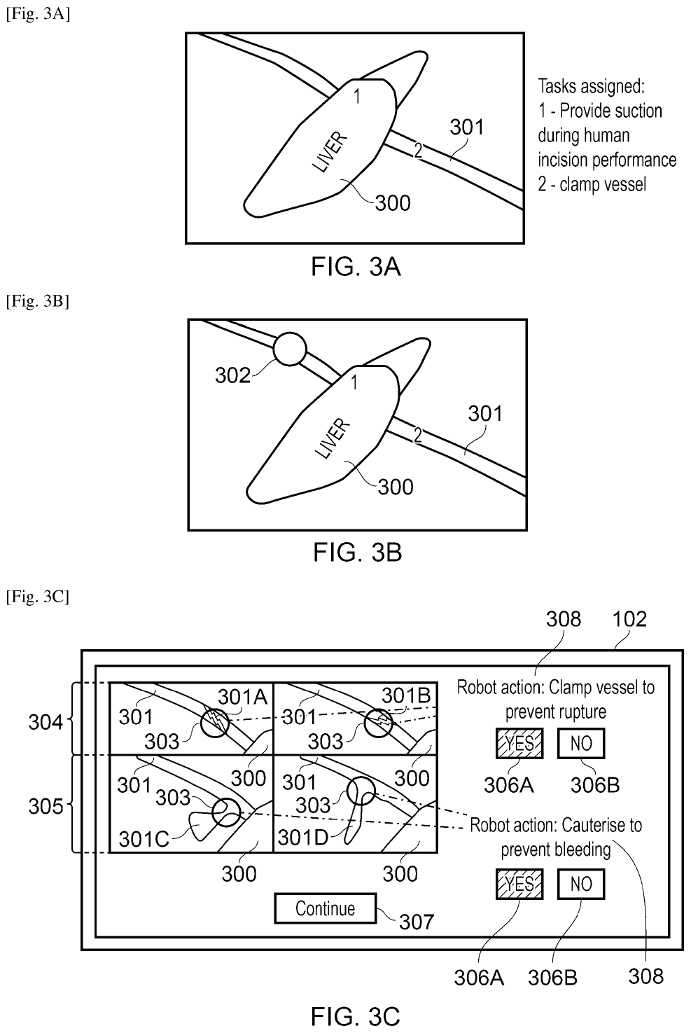

FIG. 3A shows a surgical scene as imaged by the camera 109. The scene comprises the patient's liver 300 and a blood vessel 301. Before proceeding further with the next stage of the surgery, the surgeon 104 provides tasks to the robot 103 using the user interface 209. In this case, the selected tasks are to (1) provide suction during human incision performance by the surgeon (at the section marked “1”) and (2) clamp the blood vessel (at the section marked “2”). For example, if the user interface comprises a touch screen display, the surgeon selects the tasks from a visual interactive menu provided by the user interface and selects the location in the surgical scene at which each task should be performed by selecting a corresponding location of a displayed image of the scene captured by the camera 109. In this example, the electronic display 102 is a touch screen display and therefore the user interface is comprised as part of the electronic display 102.

FIG. 3B shows a predetermined surgical scenario which may occur during the next stage of the surgical procedure. In the scenario, a vessel rupture occurs at location 302 and requires fast clamping or cauterisation by the robot 103 (e.g. using a suitable tool 107). The robot 103 is able to detect such a scenario and perform the clamping or cauterisation by classifying an image of the surgical scene captured by the camera 109 when that scenario occurs. This is possible because such an image will contain information indicating the scenario has occurred (i.e. a vessel rupture or bleed will be visually detectable in the image) and the artificial neural network used for classification by the robot 103 will, based on this information, classify the image as being an image of a vessel rupture which requires clamping or a vessel rupture which requires cauterisation. Thus, in this case, there are two possible predetermined surgical scenarios which could occur during the next stage of the surgery and which are detectable by the robot based on images captured by the camera 109. One is a vessel rupture requiring clamping (appropriate if the vessel is in the process of rupturing or has only very recently ruptured) and the other is a vessel requiring cauterisation (appropriate if the vessel has already ruptured and is bleeding).

The problem, however, is that because of the nature of artificial neural network classification, the surgeon 104 does not know what sort of images the robot 103 is looking for to detect occurrence of these predetermined scenarios. The surgeon therefore does not know how accurate the robot's determination that one of the predetermined scenarios has occurred will be and thus, conventionally, will have to give permission for the robot to perform the clamping or cauterisation if and when the relevant predetermined scenario is detected by the robot.

Prior to proceeding with the next stage of the surgery, feature visualization is therefore carried out using the image classification output by the artificial neural network to indicate the occurrence of the predetermined scenarios. Images generated using feature visualization are shown in FIG. 3C. The images are displayed on the electronic display 102. The surgeon is thus able to review the images to determine whether they are sufficiently realistic depictions of what the surgical scene would look like if each predetermined scenario (i.e. vessel rupture requiring clamping and vessel rupture requiring cauterisation) occurs.

To be clear, the images of FIG. 3C are not images of the scene captured by the camera 109. The camera 109 is still capturing the scene shown in FIG. 3A since the next stage of the surgery has not yet started. Rather, the images of FIG. 3C are artificial images of the scene generated using feature visualization of the artificial neural network based on the classification to be given to real images which show the surgical scene when each of the predetermined scenarios has occurred (the classification being possible due to training of the artificial neural network in advance using a suitable set of training images).

Each of the artificial images of FIG. 3C shows a visual feature which, if detected in a future real image captured by the camera 109, would likely result in that future real image being classified as indicating that the predetermined scenario associated with that artificial image (i.e. vessel rupture requiring clamping or vessel rupture requiring cauterisation) had occurred and that the robot 103 should therefore perform a predetermined process associated with that classification (i.e. clamping or cauterisation). In particular, a first set of artificial images 304 show a rupture 301A of the blood vessel 301 occurring in a first direction and a rupture 301B of the blood vessel 301 occurring in a second direction. These artificial images correspond to the predetermined scenario of a vessel rupture requiring clamping. The predetermined process associated with these images is therefore the robot 103 performing clamping. A second set of artificial images 305 show a bleed 301C of the blood vessel 301 having a first shape and a bleed 301D of the blood vessel 301 having a second shape. These artificial images correspond to the predetermined scenario of a vessel rupture requiring cauterisation. In both sets of images, a graphic 303 is displayed indicating the location in the image of the feature of interest, thereby helping the surgeon to easily determine the visual feature in the image likely to result in a particular classification. The location of the graphic 303 is determined based on the image feature associated with the highest level of neural network layer/neuron activation during the image visualization process, for example.

It will be appreciated that more or fewer artificial images could be generated for each set. For example, more images are generated for a more “diversified” image set (indicating possible classification for a more diverse range of image features but with reduced confidence for any specific image feature) and less images are generated for a more “optimised” image set (indicating possible classification of a less diverse range of image features but with increased confidence for any specific image feature). In an example, the number of artificial images generated using feature visualization is adjusted based on the expected visual diversity of an image feature indicating a particular predetermined scenario. Thus, a more “diverse” artificial image set may be used for a visual feature which is likely to be more visually diverse in different instances of the predetermined scenario and a more “optimised” artificial image set may be used for a visual feature which is likely to be less visually diverse in different instances of the predetermined scenario.

If the surgeon, after reviewing a set of the artificial images of FIG. 3C, determines they are a sufficiently accurate representation of what the surgical scene would look like in the predetermined scenario associated with that set, they may grant permission for the robot 103 to carry out the associated predetermined process (i.e. clamping in the case of image set 304 or cauterisation in the case of image set 305) without further permission. This will therefore occur automatically if a future image captured by the camera 109 during the next stage of the surgical procedure is classified as indicating that the predetermined scenario has occurred. The surgeon is therefore not disturbed by the robot 103 asking for permission during the surgical procedure and any time delay in the robot carrying out the predetermined process is reduced. On the other hand, if the surgeon, after reviewing a set of artificial images of FIG. 3C, determines they are not a sufficiently accurate representation of what the surgical scene would look like in the predetermined scenario associated with that set, they may not grant such permission for the robot 103. In this case, if a future image captured by the camera 109 during the next stage of the surgical procedure is classified as indicating that the predetermined scenario associated with that set has occurred, the robot will still seek permission from the surgeon before carrying out the associated predetermined process (i.e. clamping in the case of image set 304 or cauterisation in the case of image set 305). This helps ensure patient safety and reduce delays in the surgical procedure by reducing the chance that the robot 103 makes the wrong decision and thus carries out the associated predetermined process unnecessarily.

The permission (or lack of permission) is provided by the surgeon via the user interface 209. In the example of FIG. 3C, textual information 308 indicating the predetermined process associated with each set of artificial images is displayed with its respective image set, together with virtual buttons 306A and 306B indicating, respectively, whether permission is given (“Yes”) or not (“No”). The surgeon indicates whether permission is given or not by touching the relevant virtual buttons. The button most recently touched by the surgeon is highlighted (in this case, the surgeon is happy to give permission for both sets of images, and therefore the “Yes” button 306A is highlighted for both sets of images). Once the surgeon is happy with their selection, they touch the “Continue” virtual button 307. This indicates to the robot 103 that the next stage of the surgery will now begin and that images captured by the camera 109 should be classified and predetermined processes according to those classified images carried out according to the permissions selected by the surgeon.

In an embodiment, for predetermined processes not given permission in advance (e.g. if the “No” button 306B was selected for that predetermined process in FIG. 3C), permission is still requested from the surgeon during the next stage of the surgery using the electronic display 102. In this case, the electronic display simply displays textual information 308 indicating the proposed predetermined process (optionally, with the image captured by the camera 109 whose classification resulted in the proposal) and the “Yes” or “No” buttons 306A and 306B. If the surgeon selects the “Yes” button, then the robot 103 proceeds to perform the predetermined process. If the surgeon selects the “No” button, then the robot 103 does not perform the predetermined process and the surgery continues as planned.

In an embodiment, the textual information 308 indicating predetermined process to be carried out by the robot 103 may be replaced with other visual information such as a suitable graphic overlaid on the image (artificial or real) to which that predetermined process relates. For example, for the predetermined process “clamp vessel to prevent rupture” associated with the artificial image set 304 of FIG. 3C, a graphic of a clamp may be overlaid on the relevant part of each image in the set. For the predetermined process “cauterise to prevent bleeding” associated with the artificial image set 305 of FIG. 3C, a graph indicating cauterisation may be overlaid on the relevant part of each image in the set. Similar overlaid graphics may be used on a real image captured by the camera 109 in the case that advance permission is not given and thus permission from the surgeon 104 is sought during the next stage of the surgical procedure when the predetermined scenario has occurred.

In an embodiment, a surgical procedure is divided into predetermined surgical stages and each surgical stage is associated with one or more predetermined surgical scenarios. Each of the one or more predetermined surgical scenarios associated with each surgical stage is associated with an image classification of the artificial neural network such that a newly captured image of the surgical scene given that image classification by the artificial neural network is determined to be an image of the surgical scene when that predetermined surgical scenario is occurring. Each of the one or more predetermined surgical scenarios is also associated with one or more respective predetermined processes to be carried out by the robot 103 when an image classification indicates that the predetermined surgical scenario is occurring.

Information indicating the one or more predetermined surgical scenarios associated with each surgical stage and the one or more predetermined processes associated with each of those predetermined scenarios is stored in the storage medium 203. When the robot 103 is informed of the current predetermined surgical stage, it is therefore able to retrieve the information indicating the one or more predetermined surgical scenarios and the one or more predetermined processes associated with that stage and use this information to obtain permission (e.g. as in FIG. 3C) and, if necessary, perform the one or more predetermined processes.

The robot 104 is able to learn of the current predetermined surgical stage using any suitable method. For example, the surgeon 104 may inform the robot 103 of the predetermined surgical stages in advance (e.g. using a visual interactive menu system provided by the user interface 208) and, each time a new surgical stage is about to be entered, the surgeon 104 informs the robot 103 manually (e.g. by selecting a predetermined virtual button provided by the user interface 208). Alternatively, the robot 103 may determine the current surgical stage based on the tasks assigned to it by the surgeon. For example, based on tasks (1) and (2) provided to the robot in FIG. 3A, the robot may determine that the current surgical stage is that which involves the tasks (1) and (2). In this case, the information indicating each surgical stage may comprise information indicating combinations of task(s) associated with that stage, thereby allowing the robot to determine the current surgical stage by comparing the task(s) assigned to it with the task(s) associated with each surgical stage and selecting the surgical stage which has the most matching tasks. Alternatively, the robot 103 may automatically determine the current stage based on images of the surgical scene captured by the camera 109, an audio feed of the surgery captured by the microphone 113 and/or information (e.g. position, movement, operation or measurement) regarding the one or more robot tools 107, each of which will tend to have characteristics particular a given surgical stage. In an example, these characteristics may be determined using a suitable machine learning algorithm (e.g. another artificial neural network) trained using images, audio and/or tool information of a number of previous instances of the surgical procedure.

Although in the embodiment of FIGS. 3A to 3C the predetermined process is for the robot 103 to automatically perform a direct surgical action (i.e. clamping or cauterisation), the predetermined process may take the form of any other decision that can be automatically made by the robot given suitable permission. For example, the predetermined process may relate to a change of plan (e.g. altering a planned incision route) or changing the position of the camera 109 (e.g. if the predetermined surgical scenario involves blood spatter which may block the camera's view). Some other embodiments are explained below.

In one embodiment, the predetermined process performed by the robot 103 is to move the camera 109 (via control of the movable camera arm 112) to maintain a view of an active tool 107 within the surgical scene in the event that blood splatter (or splatter of another bodily fluid) might block the camera's view. In this case:

1. One of the predetermined surgical scenarios of the current surgical stage is one in which blood may spray onto the camera 109 thereby affecting the ability of the camera to image the scene.

2. Artificial images of the predetermined surgical scenario, together with information indicating the predetermined process to be carried out by the robot in the event the scenario occurs, are generated and displayed. For example:

a. Artificial images of the initial scenario or just prior to its occurrence (e.g. blood vessel incision with a scalpel and wide angle blood spray) are displayed together with an overlaid graphic (e.g. a directional arrow) indicating the robot 103 will lower the angle of incidence of the camera 109 onto the surgical scene to avoid collision with the blood spray but maintain view of the scene.

b. Artificial images of the initial scenario or just prior to its occurrence (e.g. blood vessel incision with a scalpel and wide angle blood spray) are displayed together with additional images of the same scenario where the viewpoint of the images moves in correspondence with a planned movement of the camera 109. This is achieved, for example, by mapping the artificial images onto a 3D model of the surgical scene and moving the viewpoint within the 3D model of the surgical scene to match that of the real camera in the real surgical scene (should the predetermined scenario indicating potential blood splatter occur). Alternatively, the camera 109 itself may be temporarily moved to the proposed new position and a real image captured by the camera 109 when it is in the new position displayed (thereby allowing the surgeon 104 to see the proposed different viewpoint and decide whether it is acceptable).

In one embodiment, the predetermined process performed by the robot 103 is to move the camera 109 (via control of the movable camera arm 112) to obtain the best camera angle and field of view for the current surgical stage. In this case:

1. One of the predetermined surgical scenarios of the current surgical stage is that there is a change in the surgical scene during the surgical stage for which a different camera viewing strategy is more beneficial. Example changes include:

a. Surgeon 104 switching between tools

b. Introduction of new tools

c. Retraction or removal of tools from the scene

d. Surgical stage transitions, such as revealing of a specific organ or structure which indicates that the surgery is progressing to the next stage. In this case, the predetermined surgical scenario is that the surgery is progressing to the next surgical stage.

2. Artificial images of the predetermined surgical scenario, together with information indicating the predetermined process to be carried out by the robot in the event the scenario occurs, are generated and displayed. This may involve overlaying suitable graphics indicating the direction of camera movement on the artificial images or changing the viewpoint of the artificial images or a real image as previously described. In one example, when a specific organ or structure is revealed indicating a surgical stage transition (see point (d)), the predetermined process may be to cause the camera 109 to move to a closer position with respect to the organ or structure so as to allow more precise actions to be performed on the organ or structure.

In one embodiment, the predetermined process performed by the robot 103 is to move the camera 109 (via control of the movable camera arm 112) such that one or more features of the surgical scene stay within the field of view of the camera at all times if a mistake is made by the surgeon 104 (e.g. by dropping a tool or the like). In this case:

1. One of the predetermined surgical scenarios of the current surgical stage is that a visually identifiable mistake is made by the surgeon 104. Example mistakes include:

a. Dropping a gripped organ

b. Dropping a held tool

2. Artificial images of the predetermined surgical scenario, together with information indicating the predetermined process to be carried out by the robot in the event the scenario occurs, are generated and displayed. This may involve overlaying suitable graphics indicating the direction of camera movement on the artificial images or changing the viewpoint of the artificial images or a real image as previously described. In one example, the camera position is adjusted such that the dropped item and the surgeon's hand which dropped the item are kept within the field of view of the camera all times.

In one embodiment, the predetermined process performed by the robot 103 is to move the camera 109 (via control of the movable camera arm 112) in the case that bleeding can be seen within the field of view of the camera but from a source not within the field of view. In this case:

1. One of the predetermined surgical scenarios of the current surgical stage is that there is a bleed with an unseen source.

2. Artificial images of the predetermined surgical scenario, together with information indicating the predetermined process to be carried out by the robot in the event the scenario occurs, are generated and displayed. This may involve overlaying suitable graphics indicating the direction of camera movement on the artificial images or changing the viewpoint of the artificial images or a real image as previously described. In one example, camera 109 is moved to a higher position to widen the field of view so it contains source of the bleed and the original camera focus.

In one embodiment, the predetermined process performed by the robot 103 is to move the camera 109 (via control of the movable camera arm 112) to provide an improved field of view for performance of an incision. In this case:

1. One of the predetermined surgical scenarios of the current surgical stage is that an incision is about to be performed.

2. Artificial images of the predetermined surgical scenario, together with information indicating the predetermined process to be carried out by the robot in the event the scenario occurs, are generated and displayed. This may involve overlaying suitable graphics indicating the direction of camera movement on the artificial images or changing the viewpoint of the artificial images or a real image as previously described. In one example, the camera 109 is moved directly above the patient 106 so as to provide a view of the incision with reduced tool occlusion.

In one embodiment, the predetermined process performed by the robot 103 is to move the camera 109 (via control of the moveable camera arm 112) to obtain a better view of an incision when the incision is detected as deviating from a planned incision route. In this case:

1. One of the predetermined surgical scenarios of the current surgical stage is that an incision has deviated from a planned incision path.

2. Artificial images of the predetermined surgical scenario, together with information indicating the predetermined process to be carried out by the robot in the event the scenario occurs, are generated and displayed. This may involve overlaying suitable graphics indicating the direction of camera movement on the artificial images or changing the viewpoint of the artificial images or a real image as previously described. In one example, the camera may be moved to compensate for insufficient depth resolution (or another imaging property) which caused the deviation from the planned incision route. For example, the camera may be moved to have a field of view which emphasises the spatial dimension of the deviation, thereby allowing the deviation to be more easily assessed by the surgeon.

In one embodiment, the predetermined process performed by the robot 103 is to move the camera 109 (via control of the moveable camera arm 112) to avoid occlusion (e.g. by a tool) in the camera's field of view. In this case:

1. One of the predetermined surgical scenarios of the current surgical stage is that a tool occludes the field of view of the camera.

2. Artificial images of the predetermined surgical scenario, together with information indicating the predetermined process to be carried out by the robot in the event the scenario occurs, are generated and displayed. This may involve overlaying suitable graphics indicating the direction of camera movement on the artificial images or changing the viewpoint of the artificial images or a real image as previously described. In one example, the camera is moved in an arc whilst maintaining a predetermined object of interest (e.g. incision) in its field of view so as to avoid occlusion by the tool.

In one embodiment, the predetermined process performed by the robot 103 is to move the camera 109 (via control of the moveable camera arm 112) to adjust the camera's field of view when a work area of the surgeon (e.g. as indicated by the position of a tool used by the surgeon) moves towards a boundary of the camera's field of view. In this case:

1. One of the predetermined surgical scenarios of the current surgical stage is that the work area of the surgeon approaches a boundary of the camera's current field of view.

2. Artificial images of the predetermined surgical scenario, together with information indicating the predetermined process to be carried out by the robot in the event the scenario occurs, are generated and displayed. This may involve overlaying suitable graphics indicating the direction of camera movement on the artificial images or changing the viewpoint of the artificial images or a real image as previously described. In one example, the camera is either moved to shift its field of view so the work area of the surgeon becomes central in the field of view or the field of view of the camera is expanded (e.g. by moving the camera further away or activating an optical or digital zoom out function of the camera) to keep both the surgeon's work area within the field of view (together with objects originally in the field of view).

In one embodiment, the predetermined process performed by the robot 103 is to move the camera 109 (via control of the moveable camera arm 112) to avoid a collision between the camera 109 and another object (e.g. a tool held by the surgeon). In this case:

1. One of the predetermined surgical scenarios of the current surgical stage is that the camera may collide with another object.

2. Artificial images of the predetermined surgical scenario, together with information indicating the predetermined process to be carried out by the robot in the event the scenario occurs, are generated and displayed. This may involve overlaying suitable graphics indicating the direction of camera movement on the artificial images or changing the viewpoint of the artificial images or a real image as previously described. In one example, the movement of the camera may be compensated for by implementing a digital zoom in an appropriate area of the new field of view of the camera so as to approximate the field of view of the camera before it was moved (this is possible if the previous and new fields of view of the camera have appropriate overlapping regions).

In one embodiment, the predetermined process performed by the robot 103 is to move the camera 109 (via control of the moveable camera arm 112) away from a predetermined object and towards a new event (e.g. bleeding) occurring in the camera's field of view. In this case:

1. One of the predetermined surgical scenarios of the current surgical stage is that a new event occurs within the field of view of the camera whilst the camera is focused on a predetermined object.

2. Artificial images of the predetermined surgical scenario, together with information indicating the predetermined process to be carried out by the robot in the event the scenario occurs, are generated and displayed. This may involve overlaying suitable graphics indicating the direction of camera movement on the artificial images or changing the viewpoint of the artificial images or a real image as previously described. In one example, as part of a task assigned to the robot, the camera follows the position of a needle during suturing. If there is a bleed which become visible in the field of view of the camera, the camera stops following the needle and is moved to focus on the bleed.

In the above mentioned embodiments, it will be appreciated that a change in position of the camera 109 may not always be required. Rather, it is an appropriate change of the field of view of the camera which is important. The change of the camera's field of view may or may not require a change in camera position. For example, a change in the camera's field of view may be obtained by activating an optical or digital zoom function of the camera. This changes the field of view but doesn't require the position of the camera to be physically changed. It will also be appreciated that the abovementioned embodiments could also apply to any other suitable movable and/or zoomable image capture apparatus such as a medical scope.

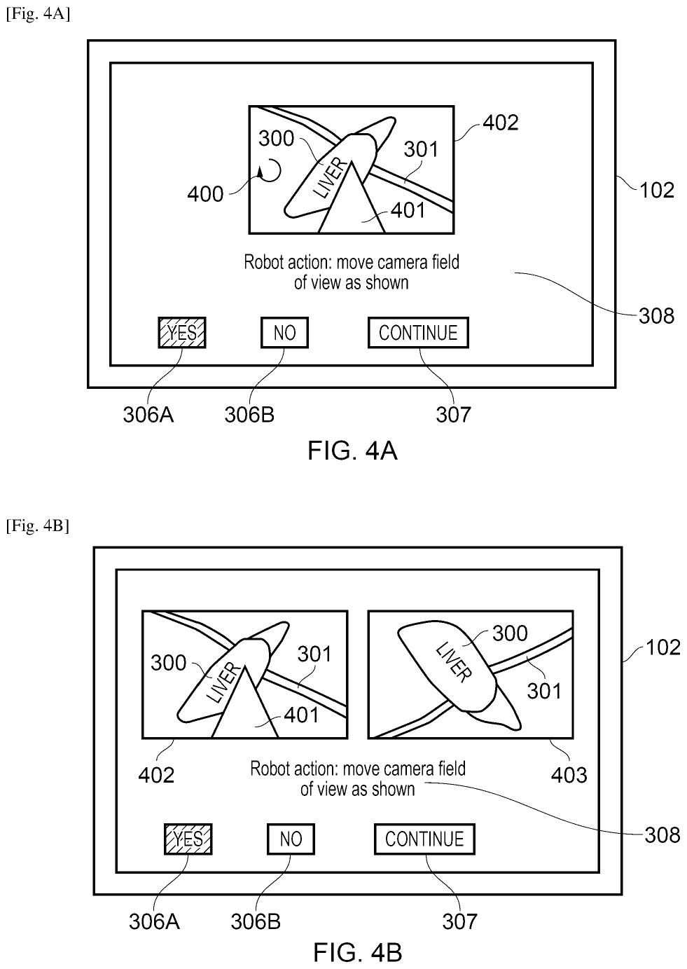

FIGS. 4A and 4B show examples of a graphic overlay or changed image viewpoint displayed on the display 102 when the predetermined process for which permission is requested relates to changing the camera's field of view. This example relates to the embodiment in which the camera's field of view is changed because a tool occludes the view of the camera 109. However, a similar arrangement may be provided for other predetermined surgical scenarios requiring a change in the camera's field of view. The display screens of FIGS. 4A and 4B are shown prior to the start of the predetermined surgical stage with which the predetermined surgical scenario is associated, for example.

FIG. 4A shows an example of a graphic overlay 400 on an artificial image 402 associated with the predetermined surgical scenario of a tool 401 occluding the field of view of the camera. The overlay 400 indicates that the predetermined process for which permission is sought is to rotate the field of view of the camera by 180 degrees whilst keeping the patient's liver 300 within the field of view. The surgeon is also informed of this by textual information 308. The surgeon reviews the artificial image 402 and determines if it is a sufficient representation of what the surgical scene would look like in the predetermined surgical scenario. In this case, the surgeon believes it is a sufficient representation. They therefore select the “Yes” virtual button 306A and then the “Continue” virtual button 307. A future classification of a real image captured by the camera during the next surgical stage which indicates the predetermined surgical scenario of a tool occluding the field of view of the camera will therefore automatically result in the position of the camera being rotated by 180 degrees whilst keeping the patient's liver 300 within the field of view. The surgeon is therefore not disturbed to give permission during the surgical procedure and occlusion of the camera's field of view by a tool is quickly alleviated.

FIG. 4B shows an example of a changed image viewpoint associated with the predetermined surgical scenario of a tool 401 occluding the field of view of the camera. The predetermined process for which permission is sought is the same as FIG. 4A, i.e. to rotate the field of view of the camera by 180 degrees whilst keeping the patient's liver 300 within the field of view. Instead of a graphic overlay on the artificial image 402, however, a further image 403 is displayed. The perspective of the further image 403 is that of the camera if it is rotated by 180 degrees according to the predetermined process. The image 403 may be another artificial image (e.g. obtained by mapping the artificial image 402 onto a 3D model of the surgical scene and rotating the field of view within the 3D model by 180 degrees according to the predetermined process). Alternatively, the image 403 may be a real image captured by temporarily rotating the camera by 180 degrees according to the predetermined process so that the surgeon is able to see the real field of view of the camera when it is in this alternative position. For example, the camera may be rotated to the proposed position long enough to capture the image 403 and then rotated back to its original position. The surgeon is again also informed of the proposed camera movement by textual information 308. The surgeon is then able to review the artificial image 402 and, in this case, again selects the “Yes” virtual button 306A and the “Continue” virtual button 307 in the same way as described for FIG. 4A.

In an embodiment, each predetermined process for which permission is sought is allocated information indicating the extent to which the predetermined process is invasive to the human patient. This is referred to as an “invasiveness score”. A more invasive predetermined process (e.g. cauterisation, clamping or an incision performed by the robot 103) is provided with a higher invasiveness score than a less invasive procedure (e.g. changing the camera's field of view). It is possible for a particular predetermined surgical scenario to be associated with multiple predetermined processes which require permission (e.g. a change of the camera field of view, an incision and a cauterisation). To reduce the time required for the surgeon to give permission for each predetermined process, if the surgeon gives permission to a predetermined process with a higher invasiveness score, permission is automatically also given to all predetermined processes with an equal or low invasiveness score. Thus, for example, if incision has the highest invasiveness score followed by cauterisation followed by changing the camera field of view, then giving permission for incision will automatically result in permission also being given for cauterisation and changing the camera field of view. Giving permission for cauterisation will automatically result in permission also being given for changing the camera field of view (but not incision, since it has a higher invasiveness score). Giving permission for changing the camera field of view will not automatically result in permission being given for cauterisation or incision (since it has a lower invasiveness score than both).

In an embodiment, following the classification of a real image captured by the camera 109 which indicates a predetermined surgical scenario has occurred, the real image is first compared with the artificial image(s) used when previously determining the permissions of the one or more predetermined processes associated with the predetermined surgical scenario. The comparison of the real image and artificial image(s) is carried out using any suitable image comparison algorithm (e.g. pixel-by-pixel comparison using suitably determined parameters and tolerances) which outputs a score indicating the similarity of two images (similarity score). The one or more predetermined processes for which permission has previously been given are then only carried out automatically if the similarity score exceeds a predetermined threshold. This helps reduce the risk of an inappropriate classification of the real image by the artificial neural network resulting in the one or more permissioned predetermined processes being carried out. Such inappropriate classification can occur, for example, if the real image comprises unexpected image features (e.g. lens artefacts or the like) with which the artificial neural network has not been trained. Although the real image does not look like the images used to train the artificial neural network to output the classification concerned, the unexpected image features can cause the artificial neural network to nonetheless output that classification. Thus, by also implementing image comparison before implementing the one or more permission predetermined processes associated with the classification, the risk of inappropriate implementation of the one or more permission predetermined processes (which could be detrimental to surgery efficiency and/or patient safety) is alleviated.

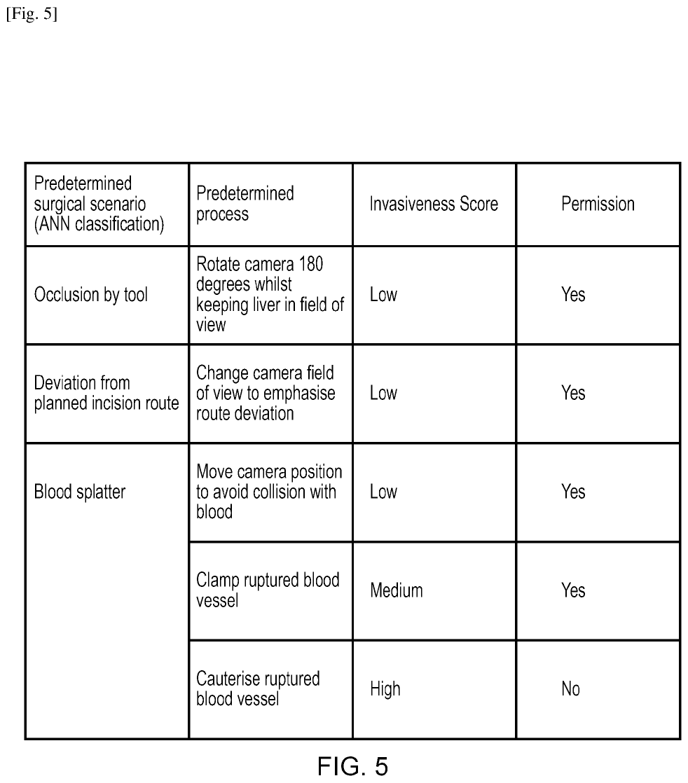

Once permission has been given (or not) for each predetermined surgical scenario associated with a particular predetermined surgical stage, information indicating each predetermined surgical scenario, the one or more predetermined processes associated with that predetermined surgical scenario and whether or not permission has been given is stored in the memory 202 and/or storage medium 203 for reference during the predetermined surgical stage. For example, the information may be stored as a lookup table like that shown in FIG. 5. The table of FIG. 5 also stores the invasiveness score (“high”, “medium” or “low”, in this example) of each predetermined process. When a real image captured by the camera is classified by the artificial neural network (ANN) as representing a predetermined surgical scenario, the processor 201 looks up the one or more predetermined processes associated with that predetermined surgical scenario and their permissions. The processor 201 then controls the robot 103 to automatically perform the predetermined processes which have been given permission (i.e. those for which the permission field is “Yes”). For those which haven't been given permission (i.e. those for which the permission field is “No”), permission will be specifically requested during the surgery and the robot 103 will not perform them unless this permission is given. The lookup table of FIG. 5 is for a predetermined surgical stage involving the surgeon making an incision on the patient's liver 300 along a predetermined route. Different predetermined surgical stages may have different predetermined surgical scenarios and different predetermined processes associated with them. This will be reflected in their respective lookup tables.

Although the above description considers a surgeon, the present technique is applicable to any human supervisor in the operating theatre (e.g. anaesthetist, nurse, etc.) whose permission must be sought before the robot 103 carries out a predetermined process automatically in a detected predetermined surgical scenario.

The present technique thus allows a supervisor of a computer assisted surgery system to give permission for actions to be carried out by a computerised surgical apparatus (e.g. robot 103) before those permissions are required. This allows permission requests to be grouped during surgery at a convenient time for the supervisor (e.g. prior to the surgery or prior to each predetermined stage of the surgery when there is less time pressure). It also allows action to be taken more quickly by the computerised surgical apparatus (since time is not wasted seeking permission when action needs to be taken) and allows the computerised surgical apparatus to handle a wider range of situations which require fast actions (where the process of requesting permission would ordinarily preclude the computerised surgical apparatus from handling the situation). The permission requests provided are also more meaningful (since the artificial images more closely represent the possible options of real stimuli which could trigger the computerised surgical apparatus to make a decision). The review effort of the human supervisor is also reduced for predetermined surgical scenarios which are likely to occur (and which would therefore conventionally require permission to be given at several times during the surgery) and for predetermined surgical scenarios which would be difficult to communicate to a human during the surgery (e.g. if decisions will need to be made quickly or require lengthy communication to the surgeon). Greater collaboration with a human surgeon is enabled where requested permissions may help to communicate to the human surgeon what the computerised surgical apparatus perceives as likely surgical scenarios.

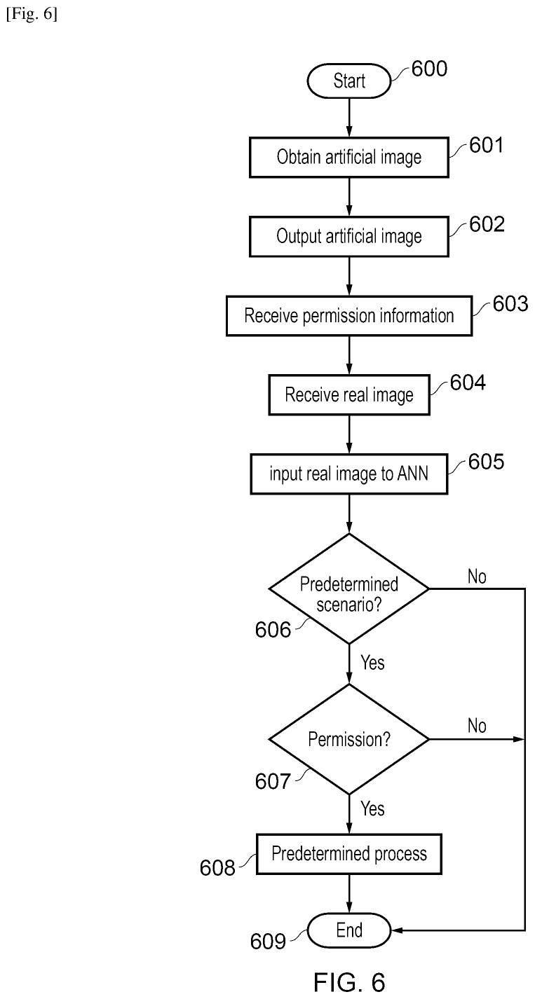

FIG. 6 shows a flow chart showing a method carried out by the controller 110 according to an embodiment.

The method starts at step 600.

At step 601, an artificial image is obtained of the surgical scene during a predetermined surgical scenario using feature visualization of the artificial neural network configured to output information indicating the predetermined surgical scenario when a real image of the surgical scene captured by the camera 109 during the predetermined surgical scenario is input to the artificial neural network.

At step 602, the display interface outputs the artificial image for display on the electronic display 102.

At step 603, the user interface 208 receives permission information indicating if a human gives permission for a predetermined process to be performed in response to the artificial neural network outputting information indicating the predetermined surgical scenario when a real image captured by the camera 109 is input to the artificial neural network.

At step 604, the camera interface 205 receives a real image captured by the camera 109.

At step 605, the real image is input to the artificial neural network.

At step 606, it is determined if the artificial neural network outputs information indicating the predetermined surgical scenario. If it does not, the method ends at step 609. If it does, the method proceeds to step 607.

At step 607, it is determined if the human gave permission for the predetermined process to be performed. If they did, the method ends at step 609. If they did, the method proceeds to step 608.

At step 608, the controller causes the predetermined process to be performed.

The process ends at step 609.

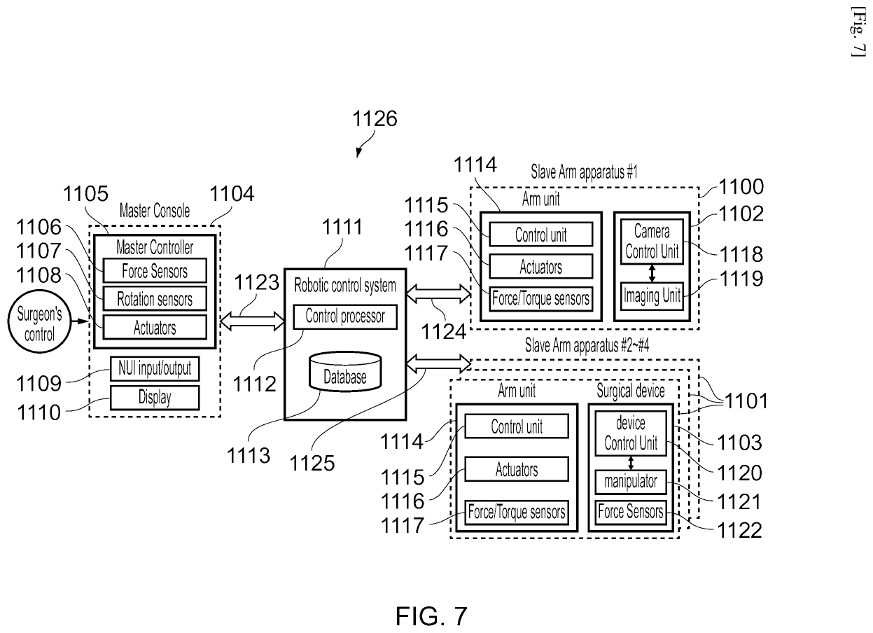

FIG. 7 schematically shows an example of a computer assisted surgery system 1126 to which the present technique is applicable. The computer assisted surgery system is a master-slave (master slave) system incorporating an autonomous arm 1100 and one or more surgeon-controlled arms 1101. The autonomous arm holds an imaging device 1102 (e.g. a surgical camera or medical vision scope such as a medical endoscope, surgical microscope or surgical exoscope). The one or more surgeon-controlled arms 1101 each hold a surgical device 1103 (e.g. a cutting tool or the like). The imaging device of the autonomous arm outputs an image of the surgical scene to an electronic display 1110 viewable by the surgeon. The autonomous arm autonomously adjusts the view of the imaging device whilst the surgeon performs the surgery using the one or more surgeon-controlled arms to provide the surgeon with an appropriate view of the surgical scene in real time.

The surgeon controls the one or more surgeon-controlled arms 1101 using a master console 1104. The master console includes a master controller 1105. The master controller 1105 includes one or more force sensors 1106 (e.g. torque sensors), one or more rotation sensors 1107 (e.g. encoders) and one or more actuators 1108. The master console includes an arm (not shown) including one or more joints and an operation portion. The operation portion can be grasped by the surgeon and moved to cause movement of the arm about the one or more joints. The one or more force sensors 1106 detect a force provided by the surgeon on the operation portion of the arm about the one or more joints. The one or more rotation sensors detect a rotation angle of the one or more joints of the arm. The actuator 1108 drives the arm about the one or more joints to allow the arm to provide haptic feedback to the surgeon. The master console includes a natural user interface (NUI) input/output for receiving input information from and providing output information to the surgeon. The NUI input/output includes the arm (which the surgeon moves to provide input information and which provides haptic feedback to the surgeon as output information). The NUI input/output may also include voice input, line of sight input and/or gesture input, for example. The master console comprises the electronic display 1110 for outputting images captured by the imaging device 1102.

The master console 1104 communicates with each of the autonomous arm 1100 and one or more surgeon-controlled arms 1101 via a robotic control system 1111. The robotic control system is connected to the master console 1104, autonomous arm 1100 and one or more surgeon-controlled arms 1101 by wired or wireless connections 1123, 1124 and 1125. The connections 1123, 1124 and 1125 allow the exchange of wired or wireless signals between the master console, autonomous arm and one or more surgeon-controlled arms.

The robotic control system includes a control processor 1112 and a database 1113. The control processor 1112 processes signals received from the one or more force sensors 1106 and one or more rotation sensors 1107 and outputs control signals in response to which one or more actuators 1116 drive the one or more surgeon controlled arms 1101. In this way, movement of the operation portion of the master console 1104 causes corresponding movement of the one or more surgeon controlled arms.

The control processor 1112 also outputs control signals in response to which one or more actuators 1116 drive the autonomous arm 1100. The control signals output to the autonomous arm are determined by the control processor 1112 in response to signals received from one or more of the master console 1104, one or more surgeon-controlled arms 1101, autonomous arm 1100 and any other signal sources (not shown). The received signals are signals which indicate an appropriate position of the autonomous arm for images with an appropriate view to be captured by the imaging device 1102. The database 1113 stores values of the received signals and corresponding positions of the autonomous arm.

For example, for a given combination of values of signals received from the one or more force sensors 1106 and rotation sensors 1107 of the master controller (which, in turn, indicate the corresponding movement of the one or more surgeon-controlled arms 1101), a corresponding position of the autonomous arm 1100 is set so that images captured by the imaging device 1102 are not occluded by the one or more surgeon-controlled arms 1101.

As another example, if signals output by one or more force sensors 1117 (e.g. torque sensors) of the autonomous arm indicate the autonomous arm is experiencing resistance (e.g. due to an obstacle in the autonomous arm's path), a corresponding position of the autonomous arm is set so that images are captured by the imaging device 1102 from an alternative view (e.g. one which allows the autonomous arm to move along an alternative path not involving the obstacle).

It will be appreciated there may be other types of received signals which indicate an appropriate position of the autonomous arm.

The control processor 1112 looks up the values of the received signals in the database 1112 and retrieves information indicating the corresponding position of the autonomous arm 1100. This information is then processed to generate further signals in response to which the actuators 1116 of the autonomous arm cause the autonomous arm to move to the indicated position.

Each of the autonomous arm 1100 and one or more surgeon-controlled arms 1101 includes an arm unit 1114. The arm unit includes an arm (not shown), a control unit 1115, one or more actuators 1116 and one or more force sensors 1117 (e.g. torque sensors). The arm includes one or more links and joints to allow movement of the arm. The control unit 1115 sends signals to and receives signals from the robotic control system 1111.

In response to signals received from the robotic control system, the control unit 1115 controls the one or more actuators 1116 to drive the arm about the one or more joints to move it to an appropriate position. For the one or more surgeon-controlled arms 1101, the received signals are generated by the robotic control system based on signals received from the master console 1104 (e.g. by the surgeon controlling the arm of the master console). For the autonomous arm 1100, the received signals are generated by the robotic control system looking up suitable autonomous arm position information in the database 1113.

In response to signals output by the one or more force sensors 1117 about the one or more joints, the control unit 1115 outputs signals to the robotic control system. For example, this allows the robotic control system to send signals indicative of resistance experienced by the one or more surgeon-controlled arms 1101 to the master console 1104 to provide corresponding haptic feedback to the surgeon (e.g. so that a resistance experienced by the one or more surgeon-controlled arms results in the actuators 1108 of the master console causing a corresponding resistance in the arm of the master console). As another example, this allows the robotic control system to look up suitable autonomous arm position information in the database 1113 (e.g. to find an alternative position of the autonomous arm if the one or more force sensors 1117 indicate an obstacle is in the path of the autonomous arm).

The imaging device 1102 of the autonomous arm 1100 includes a camera control unit 1118 and an imaging unit 1119. The camera control unit controls the imaging unit to capture images and controls various parameters of the captured image such as zoom level, exposure value, white balance and the like. The imaging unit captures images of the surgical scene. The imaging unit includes all components necessary for capturing images including one or more lenses and an image sensor (not shown). The view of the surgical scene from which images are captured depends on the position of the autonomous arm.

The surgical device 1103 of the one or more surgeon-controlled arms includes a device control unit 1120, manipulator 1121 (e.g. including one or more motors and/or actuators) and one or more force sensors 1122 (e.g. torque sensors).

The device control unit 1120 controls the manipulator to perform a physical action (e.g. a cutting action when the surgical device 1103 is a cutting tool) in response to signals received from the robotic control system 1111. The signals are generated by the robotic control system in response to signals received from the master console 1104 which are generated by the surgeon inputting information to the NUI input/output 1109 to control the surgical device. For example, the NUI input/output includes one or more buttons or levers comprised as part of the operation portion of the arm of the master console which are operable by the surgeon to cause the surgical device to perform a predetermined action (e.g. turning an electric blade on or off when the surgical device is a cutting tool).

The device control unit 1120 also receives signals from the one or more force sensors 1122. In response to the received signals, the device control unit provides corresponding signals to the robotic control system 1111 which, in turn, provides corresponding signals to the master console 1104. The master console provides haptic feedback to the surgeon via the NUI input/output 1109. The surgeon therefore receives haptic feedback from the surgical device 1103 as well as from the one or more surgeon-controlled arms 1101. For example, when the surgical device is a cutting tool, the haptic feedback involves the button or lever which operates the cutting tool to give greater resistance to operation when the signals from the one or more force sensors 1122 indicate a greater force on the cutting tool (as occurs when cutting through a harder material, e.g. bone) and to give lesser resistance to operation when the signals from the one or more force sensors 1122 indicate a lesser force on the cutting tool (as occurs when cutting through a softer material, e.g. muscle). The NUI input/output 1109 includes one or more suitable motors, actuators or the like to provide the haptic feedback in response to signals received from the robot control system 1111.

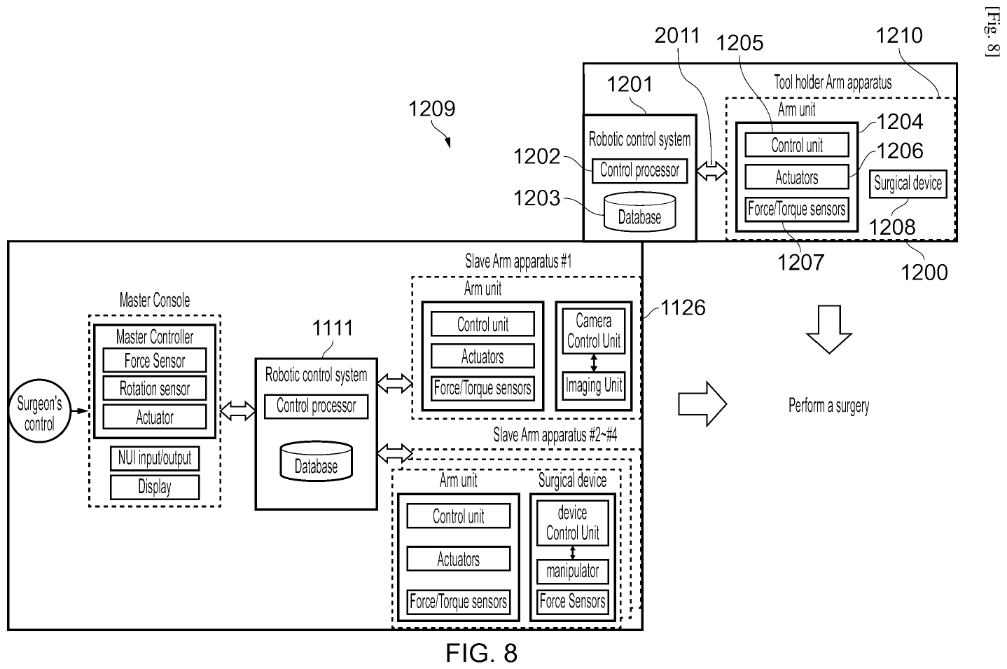

FIG. 8 schematically shows another example of a computer assisted surgery system 1209 to which the present technique is applicable. The computer assisted surgery system 1209 is a surgery system in which the surgeon performs tasks via the master-slave system 1126 and a computerised surgical apparatus 1200 performs tasks autonomously.

The master-slave system 1126 is the same as FIG. 7 and is therefore not described. The master-slave system may, however, be a different system to that of FIG. 7 in alternative embodiments or may be omitted altogether (in which case the system 1209 works autonomously whilst the surgeon performs conventional surgery).

The computerised surgical apparatus 1200 includes a robotic control system 1201 and a tool holder arm apparatus 1210. The tool holder arm apparatus 1210 includes an arm unit 1204 and a surgical device 1208. The arm unit includes an arm (not shown), a control unit 1205, one or more actuators 1206 and one or more force sensors 1207 (e.g. torque sensors). The arm comprises one or more joints to allow movement of the arm. The tool holder arm apparatus 1210 sends signals to and receives signals from the robotic control system 1201 via a wired or wireless connection 1211. The robotic control system 1201 includes a control processor 1202 and a database 1203. Although shown as a separate robotic control system, the robotic control system 1201 and the robotic control system 1111 may be one and the same. The surgical device 1208 has the same components as the surgical device 1103. These are not shown in FIG. 8.

In response to control signals received from the robotic control system 1201, the control unit 1205 controls the one or more actuators 1206 to drive the arm about the one or more joints to move it to an appropriate position. The operation of the surgical device 1208 is also controlled by control signals received from the robotic control system 1201. The control signals are generated by the control processor 1202 in response to signals received from one or more of the arm unit 1204, surgical device 1208 and any other signal sources (not shown). The other signal sources may include an imaging device (e.g. imaging device 1102 of the master-slave system 1126) which captures images of the surgical scene. The values of the signals received by the control processor 1202 are compared to signal values stored in the database 1203 along with corresponding arm position and/or surgical device operation state information. The control processor 1202 retrieves from the database 1203 arm position and/or surgical device operation state information associated with the values of the received signals. The control processor 1202 then generates the control signals to be transmitted to the control unit 1205 and surgical device 1208 using the retrieved arm position and/or surgical device operation state information.

For example, if signals received from an imaging device which captures images of the surgical scene indicate a predetermined surgical scenario (e.g. via neural network image classification process or the like), the predetermined surgical scenario is looked up in the database 1203 and arm position information and/or surgical device operation state information associated with the predetermined surgical scenario is retrieved from the database. As another example, if signals indicate a value of resistance measured by the one or more force sensors 1207 about the one or more joints of the arm unit 1204, the value of resistance is looked up in the database 1203 and arm position information and/or surgical device operation state information associated with the value of resistance is retrieved from the database (e.g. to allow the position of the arm to be changed to an alternative position if an increased resistance corresponds to an obstacle in the arm's path). In either case, the control processor 1202 then sends signals to the control unit 1205 to control the one or more actuators 1206 to change the position of the arm to that indicated by the retrieved arm position information and/or signals to the surgical device 1208 to control the surgical device 1208 to enter an operation state indicated by the retrieved operation state information (e.g. turning an electric blade to an “on” state or “off” state if the surgical device 1208 is a cutting tool).

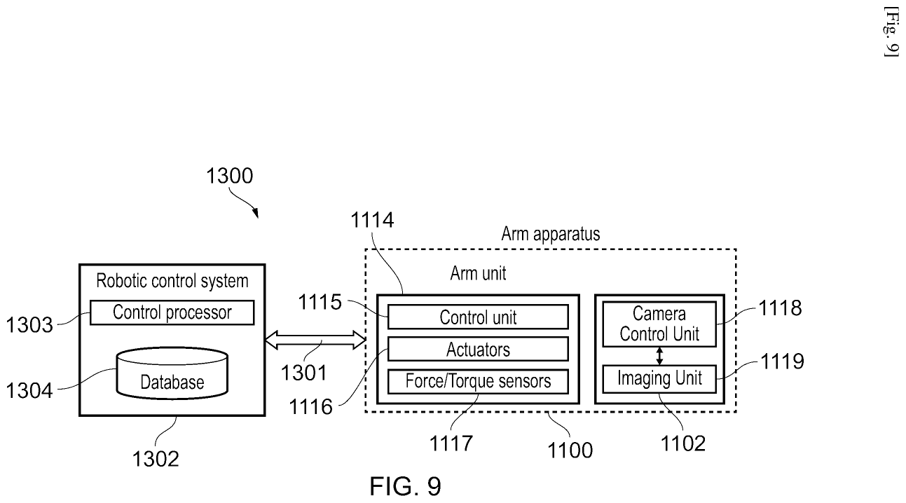

FIG. 9 schematically shows another example of a computer assisted surgery system 1300 to which the present technique is applicable. The computer assisted surgery system 1300 is a computer assisted medical scope system in which an autonomous arm 1100 holds an imaging device 1102 (e.g. a medical scope such as an endoscope, microscope or exoscope). The imaging device of the autonomous arm outputs an image of the surgical scene to an electronic display (not shown) viewable by the surgeon. The autonomous arm autonomously adjusts the view of the imaging device whilst the surgeon performs the surgery to provide the surgeon with an appropriate view of the surgical scene in real time. The autonomous arm 1100 is the same as that of FIG. 7 and is therefore not described. However, in this case, the autonomous arm is provided as part of the standalone computer assisted medical scope system 1300 rather than as part of the master-slave system 1126 of FIG. 7. The autonomous arm 1100 can therefore be used in many different surgical setups including, for example, laparoscopic surgery (in which the medical scope is an endoscope) and open surgery.

The computer assisted medical scope system 1300 also includes a robotic control system 1302 for controlling the autonomous arm 1100. The robotic control system 1302 includes a control processor 1303 and a database 1304. Wired or wireless signals are exchanged between the robotic control system 1302 and autonomous arm 1100 via connection 1301.

In response to control signals received from the robotic control system 1302, the control unit 1115 controls the one or more actuators 1116 to drive the autonomous arm 1100 to move it to an appropriate position for images with an appropriate view to be captured by the imaging device 1102. The control signals are generated by the control processor 1303 in response to signals received from one or more of the arm unit 1114, imaging device 1102 and any other signal sources (not shown). The values of the signals received by the control processor 1303 are compared to signal values stored in the database 1304 along with corresponding arm position information. The control processor 1303 retrieves from the database 1304 arm position information associated with the values of the received signals. The control processor 1303 then generates the control signals to be transmitted to the control unit 1115 using the retrieved arm position information.

For example, if signals received from the imaging device 1102 indicate a predetermined surgical scenario (e.g. via neural network image classification process or the like), the predetermined surgical scenario is looked up in the database 1304 and arm position information associated with the predetermined surgical scenario is retrieved from the database. As another example, if signals indicate a value of resistance measured by the one or more force sensors 1117 of the arm unit 1114, the value of resistance is looked up in the database 1203 and arm position information associated with the value of resistance is retrieved from the database (e.g. to allow the position of the arm to be changed to an alternative position if an increased resistance corresponds to an obstacle in the arm's path). In either case, the control processor 1303 then sends signals to the control unit 1115 to control the one or more actuators 1116 to change the position of the arm to that indicated by the retrieved arm position information.

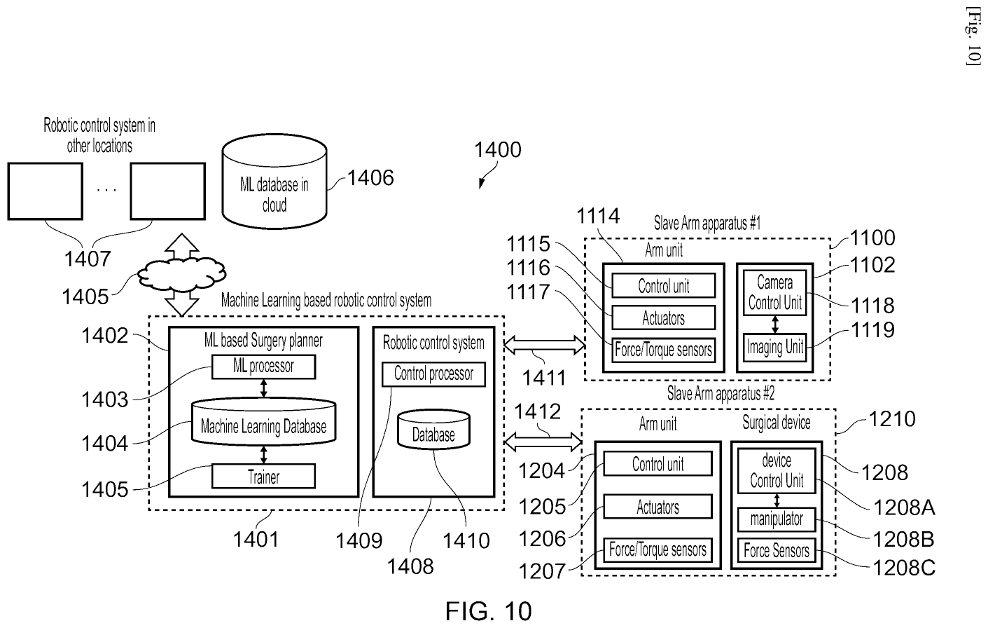

FIG. 10 schematically shows another example of a computer assisted surgery system 1400 to which the present technique is applicable. The system includes one or more autonomous arms 1100 with an imaging unit 1102 and one or more autonomous arms 1210 with a surgical device 1210. The one or more autonomous arms 1100 and one or more autonomous arms 1210 are the same as those previously described. Each of the autonomous arms 1100 and 1210 is controlled by a robotic control system 1408 including a control processor 1409 and database 1410. Wired or wireless signals are transmitted between the robotic control system 1408 and each of the autonomous arms 1100 and 1210 via connections 1411 and 1412, respectively. The robotic control system 1408 performs the functions of the previously described robotic control systems 1111 and/or 1302 for controlling each of the autonomous arms 1100 and performs the functions of the previously described robotic control system 1201 for controlling each of the autonomous arms 1210.

The autonomous arms 1100 and 1210 perform at least a part of the surgery completely autonomously (e.g. when the system 1400 is an open surgery system). The robotic control system 1408 controls the autonomous arms 1100 and 1210 to perform predetermined actions during the surgery based on input information indicative of the current stage of the surgery and/or events happening in the surgery. For example, the input information includes images captured by the image capture device 1102. The input information may also include sounds captured by a microphone (not shown), detection of in-use surgical instruments based on motion sensors comprised with the surgical instruments (not shown) and/or any other suitable input information.

The input information is analysed using a suitable machine learning (ML) algorithm (e.g. a suitable artificial neural network) implemented by machine learning based surgery planning apparatus 1402. The planning apparatus 1402 includes a machine learning processor 1403, a machine learning database 1404 and a trainer 1405.

The machine learning database 1404 includes information indicating classifications of surgical stages (e.g. making an incision, removing an organ or applying stitches) and/or surgical events (e.g. a bleed or a patient parameter falling outside a predetermined range) and input information known in advance to correspond to those classifications (e.g. one or more images captured by the imaging device 1102 during each classified surgical stage and/or surgical event). The machine learning database 1404 is populated during a training phase by providing information indicating each classification and corresponding input information to the trainer 1405. The trainer 1405 then uses this information to train the machine learning algorithm (e.g. by using the information to determine suitable artificial neural network parameters). The machine learning algorithm is implemented by the machine learning processor 1403.

Once trained, previously unseen input information (e.g. newly captured images of a surgical scene) can be classified by the machine learning algorithm to determine a surgical stage and/or surgical event associated with that input information. The machine learning database also includes action information indicating the actions to be undertaken by each of the autonomous arms 1100 and 1210 in response to each surgical stage and/or surgical event stored in the machine learning database (e.g. controlling the autonomous arm 1210 to make the incision at the relevant location for the surgical stage “making an incision” and controlling the autonomous arm 1210 to perform an appropriate cauterisation for the surgical event “bleed”). The machine learning based surgery planner 1402 is therefore able to determine the relevant action to be taken by the autonomous arms 1100 and/or 1210 in response to the surgical stage and/or surgical event classification output by the machine learning algorithm. Information indicating the relevant action is provided to the robotic control system 1408 which, in turn, provides signals to the autonomous arms 1100 and/or 1210 to cause the relevant action to be performed.

The planning apparatus 1402 may be included within a control unit 1401 with the robotic control system 1408, thereby allowing direct electronic communication between the planning apparatus 1402 and robotic control system 1408. Alternatively or in addition, the robotic control system 1408 may receive signals from other devices 1407 over a communications network 1405 (e.g. the internet). This allows the autonomous arms 1100 and 1210 to be remotely controlled based on processing carried out by these other devices 1407. In an example, the devices 1407 are cloud servers with sufficient processing power to quickly implement complex machine learning algorithms, thereby arriving at more reliable surgical stage and/or surgical event classifications. Different machine learning algorithms may be implemented by different respective devices 1407 using the same training data stored in an external (e.g. cloud based) machine learning database 1406 accessible by each of the devices. Each device 1407 therefore does not need its own machine learning database (like machine learning database 1404 of planning apparatus 1402) and the training data can be updated and made available to all devices 1407 centrally. Each of the devices 1407 still includes a trainer (like trainer 1405) and machine learning processor (like machine learning processor 1403) to implement its respective machine learning algorithm.

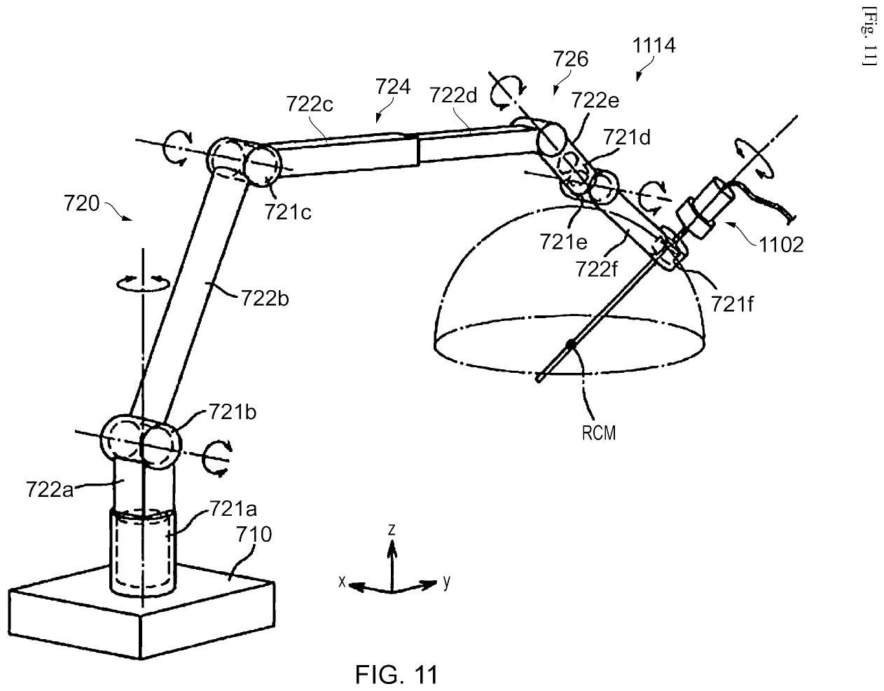

FIG. 11 shows an example of the arm unit 1114. The arm unit 1204 is configured in the same way. In this example, the arm unit 1114 supports an endoscope as an imaging device 1102. However, in another example, a different imaging device 1102 or surgical device 1103 (in the case of arm unit 1114) or 1208 (in the case of arm unit 1204) is supported.

The arm unit 1114 includes a base 710 and an arm 720 extending from the base 720. The arm 720 includes a plurality of active joints 721a to 721f and supports the endoscope 1102 at a distal end of the arm 720. The links 722a to 722f are substantially rod-shaped members. Ends of the plurality of links 722a to 722f are connected to each other by active joints 721a to 721f, a passive slide mechanism 724 and a passive joint 726. The base unit 710 acts as a fulcrum so that an arm shape extends from the base 710.

A position and a posture of the endoscope 1102 are controlled by driving and controlling actuators provided in the active joints 721a to 721f of the arm 720. According to the this example, a distal end of the endoscope 1102 is caused to enter a patient's body cavity, which is a treatment site, and captures an image of the treatment site. However, the endoscope 1102 may instead be another device such as another imaging device or a surgical device. More generally, a device held at the end of the arm 720 is referred to as a distal unit or distal device.

Here, the arm unit 700 is described by defining coordinate axes as illustrated in FIG. 11 as follows. Furthermore, a vertical direction, a longitudinal direction, and a horizontal direction are defined according to the coordinate axes. In other words, a vertical direction with respect to the base 710 installed on the floor surface is defined as a z-axis direction and the vertical direction. Furthermore, a direction orthogonal to the z axis, the direction in which the arm 720 is extended from the base 710 (in other words, a direction in which the endoscope 1102 is positioned with respect to the base 710) is defined as a y-axis direction and the longitudinal direction. Moreover, a direction orthogonal to the y-axis and z-axis is defined as an x-axis direction and the horizontal direction.

The active joints 721a to 721f connect the links to each other to be rotatable. The active joints 721a to 721f have the actuators, and have each rotation mechanism that is driven to rotate about a predetermined rotation axis by drive of the actuator. As the rotational drive of each of the active joints 721a to 721f is controlled, it is possible to control the drive of the arm 720, for example, to extend or contract (fold) the arm unit 720.

The passive slide mechanism 724 is an aspect of a passive form change mechanism, and connects the link 722c and the link 722d to each other to be movable forward and rearward along a predetermined direction. The passive slide mechanism 724 is operated to move forward and rearward by, for example, a user, and a distance between the active joint 721c at one end side of the link 722c and the passive joint 726 is variable. With the configuration, the whole form of the arm unit 720 can be changed.

The passive joint 736 is an aspect of the passive form change mechanism, and connects the link 722d and the link 722e to each other to be rotatable. The passive joint 726 is operated to rotate by, for example, the user, and an angle formed between the link 722d and the link 722e is variable. With the configuration, the whole form of the arm unit 720 can be changed.

In an embodiment, the arm unit 1114 has the six active joints 721a to 721f, and six degrees of freedom are realized regarding the drive of the arm 720. That is, the passive slide mechanism 726 and the passive joint 726 are not objects to be subjected to the drive control while the drive control of the arm unit 1114 is realized by the drive control of the six active joints 721a to 721f.

Specifically, as illustrated in FIG. 11 the active joints 721a, 721d, and 721f are provided so as to have each long axis direction of the connected links 722a and 722e and a capturing direction of the connected endoscope 1102 as a rotational axis direction. The active joints 721b, 721c, and 721e are provided so as to have the x-axis direction, which is a direction in which a connection angle of each of the connected links 722a to 722c, 722e, and 722f and the endoscope 1102 is changed within a y-z plane (a plane defined by the y axis and the z axis), as a rotation axis direction. In this manner, the active joints 721a, 721d, and 721f have a function of performing so-called yawing, and the active joints 421b, 421c, and 421e have a function of performing so-called pitching.

Since the six degrees of freedom are realized with respect to the drive of the arm 720 in the arm unit 1114, the endoscope 1102 can be freely moved within a movable range of the arm 720. FIG. 11 illustrates a hemisphere as an example of the movable range of the endoscope 723. Assuming that a central point RCM (remote centre of motion) of the hemisphere is a capturing centre of a treatment site captured by the endoscope 1102, it is possible to capture the treatment site from various angles by moving the endoscope 1102 on a spherical surface of the hemisphere in a state where the capturing centre of the endoscope 1102 is fixed at the centre point of the hemisphere.

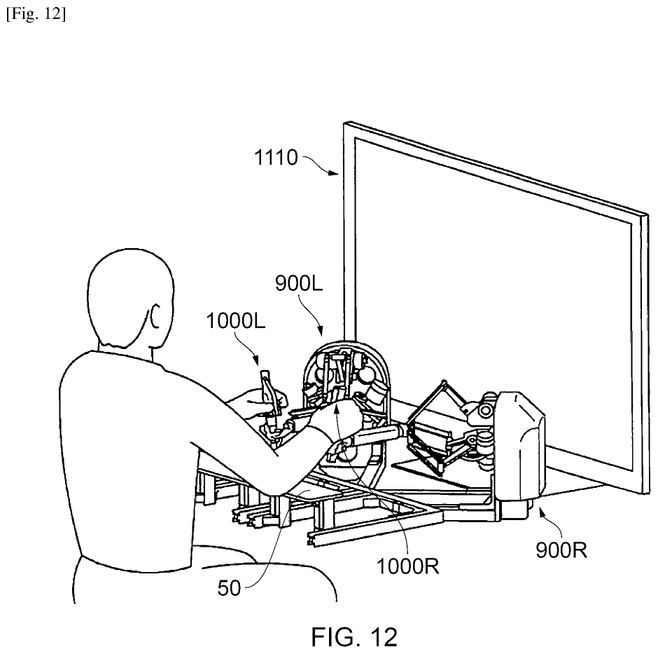

FIG. 12 shows an example of the master console 1104. Two control portions 900R and 900L for a right hand and a left hand are provided. A surgeon puts both arms or both elbows on the supporting base 50, and uses the right hand and the left hand to grasp the operation portions 1000R and 1000L, respectively. In this state, the surgeon operates the operation portions 1000R and 1000L while watching electronic display 1110 showing a surgical site. The surgeon may displace the positions or directions of the respective operation portions 1000R and 1000L to remotely operate the positions or directions of surgical instruments attached to one or more slave apparatuses or use each surgical instrument to perform a grasping operation.

Some embodiments of the present technique are defined by the following numbered clauses:

(1)
A computer assisted surgery system including an image capture apparatus, a display, a user interface and circuitry, wherein the circuitry is configured to:
receive information indicating a surgical scenario and a surgical process associated with the surgical scenario;
obtain an artificial image of the surgical scenario;
output the artificial image for display on the display;
receive permission information via the user interface indicating if there is permission for the surgical process to be performed if the surgical scenario is determined to occur.

(2)
A computer assisted surgery system according to clause 1, wherein the circuitry is configured to:
receive a real image captured by the image capture apparatus;
determine if the real image indicates occurrence of the surgical scenario;
if the real image indicates occurrence of the surgical scenario, determine if there is permission for the surgical process to be performed; and
if there is permission for the surgical process to be performed, control the predetermined process to be performed.

(3)
A computer assisted surgery system according to clause 2, wherein:
the artificial image is obtained using feature visualization of an artificial neural network configured to output information indicating the surgical scenario when a real image of the surgical scenario captured by the image capture apparatus is input to the artificial neural network; and
it is determined the real image indicates occurrence of the surgical scenario when the artificial neural network outputs information indicating the surgical scenario when the real image is input to the artificial neural network.

(4)
A computer assisted surgery system according to any preceding clause, wherein the surgical process includes controlling a surgical apparatus to perform a surgical action.

(5)
A computer assisted surgery system according to any preceding clause, wherein the surgical process includes adjusting a field of view of the image capture apparatus.

(6)
A computer assisted surgery system according to clause 5, wherein:
the surgical scenario is one in which a bodily fluid may collide with the image capture apparatus; and
the surgical process includes adjusting a position of the image capture apparatus to reduce the risk of the collision.

(7)
A computer assisted surgery system according to clause 5, wherein:
the surgical scenario is one in which there is a different field of view of the image capture apparatus is beneficial; and
the surgical process includes adjusting the field of view of the image capture apparatus to the different field of view.

(8)
A computer assisted surgery system according to clause 7, wherein:
the surgical scenario is one in which an incision is performed; and
the different field of view provides an improved view of the performance of the incision.

(9)
A computer assisted surgery system according to clause 8, wherein:
the surgical scenario includes the incision deviating from the planned incision; and
the different field of view provides an improved view of the deviation.

(10)
A computer assisted surgery system according to clause 5, wherein:
the surgical scenario is one in which an item is dropped; and
the surgical process includes adjusting the field of view of the image capture apparatus to keep the dropped item within the field of view.

(11)
A computer assisted surgery system according to clause 5, wherein:
the surgical scenario is one in which there is evidence within the field of view of the image capture apparatus of an event not within the field of view; and
the surgical process includes adjusting the field of view of the image capture apparatus so that the event is within the field of view.

(12)
A computer assisted surgery system according to clause 11, wherein the event is a bleed.

(13)
A computer assisted surgery system according to clause 5, wherein:
the surgical scenario is one in which an object occludes the field of view of the image capture apparatus; and
the surgical process includes adjusting the field of view of the image capture apparatus to avoid the occluding object.

(14)
A computer assisted surgery system according to clause 5, wherein:
the surgical scenario is one in which a work area approaches a boundary of the field of view of the image capture apparatus; and
the surgical process includes adjusting the field of view of the image capture apparatus so that the work area remains within the field of view.

(15)
A computer assisted surgery system according to clause 5, wherein:
the surgical scenario is one in which the image capture apparatus may collide with another object; and
the surgical process includes adjusting a position of the image capture apparatus to reduce the risk of the collision.

(16)
A computer assisted surgery system according to clause 2 or 3, wherein the circuitry is configured to:
compare the real image to the artificial image; and
perform the surgical process if a similarity between the real image and artificial image exceeds a predetermined threshold.

(17)
A computer assisted surgery system according to any preceding clause, wherein:
the surgical process is one of a plurality of surgical processes performable if the surgical scenario is determined to occur;
each of the plurality of surgical processes is associated with a respective level of invasiveness; and
each surgical process other than the surgical process is given permission to be performed if the surgical process is given permission to be performed if a level of invasiveness of that other surgical process is less than or equal to the level of invasiveness of the surgical process.

(18)
A computer assisted surgery system according to any preceding clause, wherein the image capture apparatus is a surgical camera or medical vision scope.

(19)
A computer assisted surgery system according to any preceding clause, wherein the computer assisted surgery system is a computer assisted medical vision scope system, a master-slave system or an open surgery system.

(20)
A surgical control apparatus including circuitry configured to:
receive information indicating a surgical scenario and a surgical process associated with the surgical scenario;
obtain an artificial image of the surgical scenario;
output the artificial image for display on a display;
receive permission information via a user interface indicating if there is permission for the surgical process to be performed if the surgical scenario is determined to occur.

(21)
A surgical control method including:
receiving information indicating a surgical scenario and a surgical process associated with the surgical scenario obtaining an artificial image of the surgical scenario;
outputting the artificial image for display on a display;
receiving permission information via a user interface indicating if there is permission for the surgical process to be performed if the surgical scenario is determined to occur.

(22)
A program for controlling a computer to perform a surgical control method according to clause 21.

(23)
A non-transitory storage medium storing a computer program according to clause 22.

Numerous modifications and variations of the present disclosure are possible in light of the above teachings. It is therefore to be understood that within the scope of the appended claims, the disclosure may be practiced otherwise than as specifically described herein.

In so far as embodiments of the disclosure have been described as being implemented, at least in part, by software-controlled data processing apparatus, it will be appreciated that a non-transitory machine-readable medium carrying such software, such as an optical disk, a magnetic disk, semiconductor memory or the like, is also considered to represent an embodiment of the present disclosure.

It will be appreciated that the above description for clarity has described embodiments with reference to different functional units, circuitry and/or processors. However, it will be apparent that any suitable distribution of functionality between different functional units, circuitry and/or processors may be used without detracting from the embodiments.

Described embodiments may be implemented in any suitable form including hardware, software, firmware or any combination of these. Described embodiments may optionally be implemented at least partly as computer software running on one or more data processors and/or digital signal processors. The elements and components of any embodiment may be physically, functionally and logically implemented in any suitable way. Indeed the functionality may be implemented in a single unit, in a plurality of units or as part of other functional units. As such, the disclosed embodiments may be implemented in a single unit or may be physically and functionally distributed between different units, circuitry and/or processors.

Although the present disclosure has been described in connection with some embodiments, it is not intended to be limited to the specific form set forth herein. Additionally, although a feature may appear to be described in connection with particular embodiments, one skilled in the art would recognize that various features of the described embodiments may be combined in any manner suitable to implement the technique.

1. A computer assisted surgery system comprising an image capture apparatus, a display, a user interface and circuitry, wherein the circuitry is configured to:
receive information indicating a surgical scenario and a surgical process associated with the surgical scenario;
obtain an artificial image of the surgical scenario;
output the artificial image for display on the display;
receive permission information via the user interface indicating if there is permission for the surgical process to be performed if the surgical scenario is determined to occur.

2. A computer assisted surgery system according to claim 1, wherein the circuitry is configured to:
receive a real image captured by the image capture apparatus;
determine if the real image indicates occurrence of the surgical scenario;
if the real image indicates occurrence of the surgical scenario, determine if there is permission for the surgical process to be performed; and
if there is permission for the surgical process to be performed, control the predetermined process to be performed.

3. A computer assisted surgery system according to claim 2, wherein:
the artificial image is obtained using feature visualization of an artificial neural network configured to output information indicating the surgical scenario when a real image of the surgical scenario captured by the image capture apparatus is input to the artificial neural network; and
it is determined the real image indicates occurrence of the surgical scenario when the artificial neural network outputs information indicating the surgical scenario when the real image is input to the artificial neural network.

4. A computer assisted surgery system according to claim 1, wherein the surgical process comprises controlling a surgical apparatus to perform a surgical action.

5. A computer assisted surgery system according to claim 1, wherein the surgical process comprises adjusting a field of view of the image capture apparatus.

6. A computer assisted surgery system according to claim 5, wherein:
the surgical scenario is one in which a bodily fluid may collide with the image capture apparatus; and
the surgical process comprises adjusting a position of the image capture apparatus to reduce the risk of the collision.

7. A computer assisted surgery system according to claim 5, wherein:
the surgical scenario is one in which there is a different field of view of the image capture apparatus is beneficial; and
the surgical process comprises adjusting the field of view of the image capture apparatus to the different field of view.

8. A computer assisted surgery system according to claim 7, wherein:
the surgical scenario is one in which an incision is performed; and
the different field of view provides an improved view of the performance of the incision.

9. A computer assisted surgery system according to claim 8, wherein:
the surgical scenario comprises the incision deviating from the planned incision; and
the different field of view provides an improved view of the deviation.

10. A computer assisted surgery system according to claim 5, wherein:
the surgical scenario is one in which an item is dropped; and
the surgical process comprises adjusting the field of view of the image capture apparatus to keep the dropped item within the field of view.

11. A computer assisted surgery system according to claim 5, wherein:
the surgical scenario is one in which there is evidence within the field of view of the image capture apparatus of an event not within the field of view; and
the surgical process comprises adjusting the field of view of the image capture apparatus so that the event is within the field of view.

12. A computer assisted surgery system according to claim 11, wherein the event is a bleed.

13. A computer assisted surgery system according to claim 5, wherein:
the surgical scenario is one in which an object occludes the field of view of the image capture apparatus; and
the surgical process comprises adjusting the field of view of the image capture apparatus to avoid the occluding object.

14. A computer assisted surgery system according to claim 5, wherein:
the surgical scenario is one in which a work area approaches a boundary of the field of view of the image capture apparatus; and
the surgical process comprises adjusting the field of view of the image capture apparatus so that the work area remains within the field of view.

15. A computer assisted surgery system according to claim 5, wherein:
the surgical scenario is one in which the image capture apparatus may collide with another object; and
the surgical process comprises adjusting a position of the image capture apparatus to reduce the risk of the collision.

16. A computer assisted surgery system according to claim 2, wherein the circuitry is configured to:
compare the real image to the artificial image; and
perform the surgical process if a similarity between the real image and artificial image exceeds a predetermined threshold.

17. A computer assisted surgery system according to claim 1, wherein:
the surgical process is one of a plurality of surgical processes performable if the surgical scenario is determined to occur;
each of the plurality of surgical processes is associated with a respective level of invasiveness; and
each surgical process other than the surgical process is given permission to be performed if the surgical process is given permission to be performed if a level of invasiveness of that other surgical process is less than or equal to the level of invasiveness of the surgical process.

18. A computer assisted surgery system according to claim 1, wherein the image capture apparatus is a surgical camera or medical vision scope.

19. A computer assisted surgery system according to claim 1, wherein the computer assisted surgery system is a computer assisted medical vision scope system, a master-slave system or an open surgery system.

20. A surgical control apparatus comprising circuitry configured to:
receive information indicating a surgical scenario and a surgical process associated with the surgical scenario;
obtain an artificial image of the surgical scenario;
output the artificial image for display on a display;
receive permission information via a user interface indicating if there is permission for the surgical process to be performed if the surgical scenario is determined to occur.

21. A surgical control method comprising:
receiving information indicating a surgical scenario and a surgical process associated with the surgical scenario obtaining an artificial image of the surgical scenario;
outputting the artificial image for display on a display;
receiving permission information via a user interface indicating if there is permission for the surgical process to be performed if the surgical scenario is determined to occur.

22. A program for controlling a computer to perform a surgical control method according to claim 21.

23. A non-transitory storage medium storing a computer program according to claim 22.
